(19) United States
(12) Patent Application Publication
(10) Pub. No.: US 2001/0018043 A1
(43) Pub. Date: Aug. 30, 2001

(54) Oral and dental care compositions

(75) Inventors: Torsten Henning, Kelkheim (DE); Angelika Turowski-Wanke, Kelkheim (DE); Wiebke Johannpeter, Steinbach (DE)

Correspondence Address: CLARIANT CORPORATION, 4331 CHESAPEAKE DR, ATTN: INDUSTRIAL PROPERTY DEPT, CHARLOTTE, NC, 28216, US

(73) Assignee: Clariant GmbH

(21) Appl. No.: 09/780,217

(22) Filed: Feb. 9, 2001

(30) Foreign Application Priority Data
Feb. 10, 2000 (DE) 10005831.0

(51) Int. Cl. A61K 7/16; C07C 59/235

(52) U.S. Cl. 424/49; 554/227

(57) ABSTRACT

Oral and dental care compositions comprising sorbitol esters obtained by transesterification of optionally oxalkylated sorbitol with fatty acid methyl esters or fatty acid triglycerides and optionally oxalkylation of the reaction products obtained by transesterification with fatty acid methyl esters.

### BACKGROUND OF THE INVENTION

The invention relates to surface-active compositions, in particular oral and dental care compositions, with a selected content of sorbitol esters. The products have excellent taste and odor quality and are dermatologically very compatible.

DE 199 06 368.0 describes mixtures of open-chain sorbitol monoesters, diesters and partial glycerides with small amounts of cyclic sorbitol ester fractions, which are very effective in lowering the interfacial tension of both polar and nonpolar liquids, effect high stability of the emulsions even under thermal stress, exhibit low sensitivity toward electrolytes and aids and are very kind to the skin.

### SUMMARY OF THE INVENTION

Surprisingly, we have found that the sorbitol esters described therein, which are prepared by transesterification of optionally oxalkylated sorbitol with fatty acid methyl esters or fatty acid triglycerides and optionally oxalkylation of the reaction products obtained by transesterification with fatty acid methyl esters, are excellent solubilizers of aroma substances and perfume oils in dental and oral care products and, moreover, act as consistency regulators. An essential advantage of the sorbitol esters used according to the invention in oral and dental care compositions is their excellent taste and odor neutrality.

The invention provides oral and dental care compositions comprising open-chain sorbitol esters prepared by transesterification of optionally oxalkylated sorbitol with fatty acid methyl esters or fatty acid triglycerides and optionally oxalkylation of the reaction products obtained by transesterification with fatty acid methyl esters.

### DESCRIPTION OF THE PREFERRED EMBODIMENTS

The sorbitol esters used according to the invention in oral and dental care compositions are prepared by reaction of sorbitol with fatty acid methyl esters or fatty acid triglycerides in accordance with the process described in DE 197 27 950, the catalysts being used here, however, being customary alkaline catalysts, in particular sodium methoxide. The fatty acid radical in the fatty acid methyl esters and the fatty acid triglycerides generally contains 8 to 22 carbon atoms and can be straight-chain or branched, saturated or unsaturated. Examples thereof are palmitic acid, stearic acid, lauric acid, linoleic acid, linolenic acid, isostearic acid or oleic acid. Suitable fatty acid triglycerides are all native animal or vegetable oils, fats and waxes, for example olive oil, rapeseed oil, palm kernel oil, sunflower oil, coconut oil, linseed oil, castor oil, soybean oil, optionally also in refined or hydrogenated form. Since these natural fats, oils and waxes are normally mixtures of fatty acids having different chain lengths, this also applies for the fatty acid radicals in the sorbitol esters used according to the invention.

The reaction of sorbitol with the fatty acid triglycerides or methyl esters is carried out in a one-pot process without solvents at temperatures of approximately 120-140° C. in the presence of an alkaline catalyst. The reaction time is generally 12 to 13 hours. If fatty acid methyl esters are used, the methanol which forms is distilled off during this reaction. Since sorbitol is usually available commercially as an aqueous solution, it is necessary to firstly remove the water. This is effected by distillation at a maximum of 120° C. under reduced pressure. The molar ratio of sorbitol to fatty acid methyl ester is generally 1:1 to 1:2. If fatty acid triglycerides are used, the molar ratio is generally 1 mol of sorbitol per 3.5 to 4.5 mol of fatty acid triglyceride.

The reaction products may also be alkoxylated, preferably ethoxylated, and the content of ethoxylate groups can be from 1 to 90 —CH₂CH₂O groups per molecule of sorbitol. The alkoxylate groups can be introduced by means of an alkoxylation of the sorbitol by processes known per se prior to the transesterification reaction. In the case of the transesterification with fatty acid methyl esters, preference is, however, given to a procedure in which the transesterification is firstly carried out and then, by processes known per se, the alkoxylate groups are introduced.

In addition to residual amounts of unreacted sorbitol, the reaction product of this transesterification reaction consists essentially of the sorbitol monofatty acid esters and the sorbitol difatty acid esters. The corresponding triesters are only formed in minor amounts. If fatty acid triglycerides are used as the starting material, the reaction product also comprises mono- and difatty acid glyceride and also unreacted triglyceride depending on the molar ratio of the starting compounds chosen in each case.

The resulting mixture of the different reaction products is highly suitable as a solubilizing agent, in particular of aroma components, and as bodying agents in oral and dental care compositions, in particular toothpastes and tooth cleaning gels, mouthwashes and denture cleaners.

The agents used according to the invention are added to the oral and dental care compositions in the amounts by weight of from 0.1 to 10%, preferably 0.5 to 5%, particularly preferably 1 to 3%.

In addition to comprising said sorbitol esters, the oral and dental care compositions according to the invention may comprise flavoring and aroma substances, nonionic and/or anionic surfactants, abrasives and polishes, sweeteners, humectants, consistency regulators, deodorizing active ingredients, active ingredients for oral and dental disorders, and water-soluble fluorine compounds.

Flavoring and aroma substances suitable for oral and dental care compositions are, for example, peppermint oil, spearmint oil, aniseed oil, star anise oil, caraway oil, eucalyptus oil, fennel, cinnamon oil, oil of cloves, geranium oil, sage oil, pimento oil, thyme oil, marjoram oil, basil oil, citrus oil, wintergreen oil or one or more components of these oils isolated therefrom or produced synthetically, such as e.g. menthol, carvone, anethole, cineol, eugenol, cinnamaldehyde, caryophyllene, geraniol, citronellol, linalool, salvene, thymol, terpinene, terpineol, methylchavicol and methyl salicylate. Further suitable aromas are e.g. methyl acetate, vanillin, ionones, linalyl acetate, rhodinol and piperitone.

The compositions according to the invention comprise aroma components in the amounts by weight of from 0.1 to 10%, preferably 0.5 to 5%, particularly preferably 0.8 to 2%, based on the finished composition.

Suitable nonionic surfactants are alkyl and/or alkenyl oligoglycosides, fatty acid N-alkylpolyhydroxyalkylamides, in particular fatty acid N-methylglycoside, sucrose esters, fatty alcohol polyglycol ethers, alkylphenol polyglycol ethers, fatty acid polyglycol esters, fatty acid amide polyglycol ethers, fatty amine polyglycol ethers, alkoxylated triglycerides, fatty acid glucamides, polyol fatty acid esters, sugar esters, sorbitan esters and polysorbates and/or alkoxylated fatty alcohols.

Preferred anionic surfactants are alkyl sulfates, alkenyl sulfates, alkylbenzene-sulfonates, alkanesulfonates and sec. alkanesulfonates, olefinsulfonates, alkyl ether sulfonates, glycerol ether sulfonates, α-methyl ester sulfonates, sulfo fatty acids, fatty alcohol ether sulfates, glycerol ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, acyl lactates, alkyl oligoglucoside sulfates and alkyl (ether) phosphates, C₈-C₂₀-fatty acid alpha-methyl ester sulfonates and alkyl ether sulfates.

The compositions according to the invention may comprise surfactants in the amounts by weight of from 0.1 to 12%, preferably 0.5 to 8%, particularly preferably 1 to 3%, based on the finished composition.

As abrasives and polishes, the compositions according to the invention may comprise chalk, dicalcium phosphate, insoluble sodium metaphosphate, aluminum silicate, phyllosilicates, hydrocalcites, calcium pyrophosphate, finely divided synthetic resins, silicas, aluminum oxide, aluminum oxide trihydrate, talc, zeolites, magnesium aluminum silicate, calcium sulfate, magnesium carbonate and/or magnesium oxide.

Abrasives and polishes may be present in the compositions according to the invention in the amounts by weight of from 2 to 40%, preferably 5 to 30%, particularly preferably 8 to 15%.

Suitable sweeteners are natural sugars, such as sucrose, maltose, lactose, dextrose and fructose, or synthetic sweeteners, such as e.g. saccharin sodium salt, sodium cyclamate, D-tryptophan or aspartame.

Sweeteners are used in the compositions according to the invention in amounts by weight of from 0.005 to 2%.

Suitable humectants are sorbitol, glycerol and polyglycols, for example polyglycols 200 USP to 4000. Humectants are used in the compositions according to the invention in the amounts by weight of from 1 to 95%, preferably 30 to 65%.

The compositions according to the invention are notable for an excellent taste and odor quality and are dermatologically very compatible. The examples below serve to illustrate the subject-matter of the invention in more detail, without limiting it thereto.

### EXAMPLES

### Examples

Tooth cleaning gel

| | | |
|---|---|---|
| ® Tylose C 300 P2 | 0.40 | |
| Sorbitol (70% in water) | 70.00 | |
| ® Emulsogen SRO | 2.00 | |
| PEG-32 | 3.00 | |
| Water | 4.64 | |
| Sodium saccharin | 0.07 | |
| Sodium fluoride | 0.24 | |
| Aroma substance | 1.10 | |
| PHB ethyl ester | 0.15 | |
| Phyllosilicate/abrasive | 11.00 | |
| ® Carbopol 980 | 6.00 | |
| Sodium lauryl sulfate | 1.40 | |
| 2 in 1 toothpaste | | |
| PEG-8 | 3.0 | A |
| Xanthan gum | 0.5 | |
| Sorbitol (70% in water) | 15.00 | |
| Glycerol | 55.00 | |
| Water | 10.86 | |
| Phyllosilicate/abrasive | 9.00 | |
| Sweetener | 0.18 | |
| Preservative | 0.2 | |
| Dye | 1.25 | |
| Sodium lauryl sulfate | 1.2 | |
| Emulsogen SRO | 3.00 | B |
| Aroma substance | 0.80 | |
| Menthol | 0.01 | |

Incorporate aroma into Emulsogen SRO and then stir into the mixture A under reduced pressure and homogenize.

Gel toothpaste

| | | |
|---|---|---|
| Sorbitol (70% in water) | 43.00 | A |
| Emulsogen SRO | 2.00 | |
| PEG-32 | 5.00 | |
| Sodium monofluorophosphate | 1.12 | |
| Sweetener | 0.20 | |
| Trisodium phosphate | 0.10 | |
| Water | 22.15 | |
| Dissolve A and add to the mixer | | |
| Abrasive: silicate | 10.00 | B |
| Silica gel | 8.00 | |
| Tylose C 300 P2 | 0.90 | |
| Titanium dioxide | 0.50 | |

Premix B and add to the mixer Mix for 15 minutes at 25 to 30° C. under reduced pressure. Release vacuum.

| | | |
|---|---|---|
| Sodium lauryl sulfate | 1.50 | C |
| Water | 5.53 | |

Dissolve C and add to the mixer

All percentages in Application Examples 1 to 6 are percentages by weight.

List of products used

Tylose C 300 P2: carboxymethylcellulose

Carbopol 980: polyacrylic acid

The product Emulsogen SRO (sorbitol ester of rapeseed oil) was prepared as follows:

1 mol of sorbitol in the form of a 70% strength sorbitol syrup were initially introduced, and the water was distilled off at 120° C. in a water-pump vacuum. Following the addition of 1% by weight (based on the total initial weight) of potassium carbonate (30% strength in water at 80° C.), the water was distilled off under a complete water-pump vacuum. 4 mol of refined rapeseed oil were then added and the mixture was stirred at 140° C. for 8 hours.

1. An oral and dental care composition comprising sorbitol esters obtained by transesterification of optionally oxalkylated sorbitol with fatty acid methyl esters or fatty acid triglycerides and optionally oxalkylation of the reaction products obtained by transesterification with fatty acid methyl esters.

2. The oral and dental care composition as claimed in claim 1, comprising sorbitol esters obtained by reaction of sorbitol with fatty acid triglycerides.

3. The oral and dental care composition as claimed in claim 1, which comprises sorbitol esters in the amounts by weight of from 0.1% to 10%, preferably 0.5 to 5%, particularly preferably 1 to 3%.

4. The use of sorbitol esters obtained by transesterification of optionally oxalkylated sorbitol with fatty acid methyl esters or fatty acid triglycerides and optionally oxalkylation of the reaction products obtained by transesterification with fatty acid methyl esters.
